(19) United States
(12) Patent Application Publication
(10) Pub. No.: US 2025/0235611 A1
(43) Pub. Date: Jul. 24, 2025

(54) INSERTER DEVICES INCLUDING REUSABLE AND DISPOSABLE PORTIONS

(71) Applicant: Eli Lilly and Company, Indianapolis, IN (US)

(72) Inventors: Mary Teresa CARTER, Boston, MA (US); Louise Emma VAN DEN HEUVEL, Medford, MA (US)

(21) Appl. No.: 18/697,788

(22) PCT Filed: Oct. 28, 2022
(86) PCT No.: PCT/US2022/048131

§ 371 Date: Apr. 2, 2024

Related U.S. Application Data
(60) Provisional application No. 63/273,263, filed on Oct. 29, 2021.

(51) Int. Cl. A61M 5/158; A61M 5/14

(52) U.S. Cl. CPC A61M 5/158; A61M 5/1413; A61M 2005/1585; A61M 2005/1586

(57) ABSTRACT

A shift head inserter device is disclosed that provides automatic insertion of an insertion needle and cannula and subsequent automatic retraction of the insertion needle. The inserter device is partially reusable.

### FIELD OF THE DISCLOSURE

The present disclosure relates to drug infusion systems, more specifically an inserter device that can be used to insert a cannula into the subcutaneous space of a patient, and methods of using the same.

### BACKGROUND

Infusion sets are used to deliver a drug to the subcutaneous space of a patient. The head assembly of the infusion set has a fluid path in the form of a stainless-steel needle or soft cannula that must be inserted to the correct depth in the subcutaneous tissue. To insert a soft cannula, a stainless-steel insertion needle is often used. The insertion needle opens a hole in the tissue to allow the cannula to enter and provides stiffness for the cannula as it is inserted. After insertion, the insertion needle is removed.

The insertion and retraction of the insertion needle constitute separate steps which may be performed manually by a user or automated through an inserter (that is, the inserter providing one or more forces to move the components thereof). Most commercially-available inserters automate insertion of the insertion needle. After insertion, the insertion needle is typically retracted manually. Other commercially-available inserters provide manual insertion of the insertion needle and, after insertion, the insertion needle is automatically retracted.

Another consideration is which components move during insertion. Most inserters are “shift head” devices 1000 (FIG. 1), in which the fluid path 1002 is permanently coupled to the infusion head 1004 and travels with the infusion head 1004 during the insertion process as an integrated infusion head assembly 1006. In contrast, other inserters are “shift fluid path” devices 2000 (FIG. 2), in which the fluid path 2002 is initially separated from the infusion head 2004 and is advanced toward and joined to the infusion head 2004 during the insertion process.

Shift head devices 1000 illustrated in FIG. 1 and shift fluid path devices 2000 illustrated in FIG. 2 each have various advantages over each other. For example, shift head devices 1000 may be more mechanically robust because the entire infusion head assembly 1006 moves together. Shifting the entire infusion head assembly 1006 also may facilitate omitting gaps between components that might allow water ingress, which in turn reduces infection risks. In contrast, for shift fluid path devices 2000 (FIG. 2), the infusion head 2004 and an adhesive patch are initially attached to the patient's skin when the device 2000 is positioned on the skin. When the patient or the patient's caregiver activates the device 2000, the fluid path 2002 shifts towards the skin and couples to the infusion head 2004. Because the adhesive patch and infusion head 2004 are initially attached to the skin, shift fluid path devices 2000 may be smaller and more compact. However, such devices have additional mechanical complexity due to the increased number of components and the dynamic interface. Coupling the fluid path 2002 into the infusion head 2004 must also be controlled to minimize unwanted crevices near the insertion site. To capture the fluid path 2002, clips 2006 of the infusion head 2004 may have some tolerance to them; however—even if captured correctly—the fluid path 2002 may wobble slightly in clips 2006 due to the tolerance, potentially causing discomfort. Taken as a whole, shift head devices provide several advantages over shift fluid path devices.

In view of the various drawbacks of commercially-available inserters, improved inserter devices are needed.

### SUMMARY

The present disclosure relates to a shift head inserter device that provides automatic insertion of an insertion needle and cannula and subsequent automatic retraction of the insertion needle. In addition, the inserter device is partially reusable.

According to an exemplary embodiment of the present disclosure, an inserter device is disclosed including a disposable portion and a reusable portion that is configured to detachably couple to the disposable portion. The disposable portion includes a disposable housing; an insertion needle; and an infusion cannula detachably coupled to the disposable housing and the insertion needle. The infusion cannula is configured to be inserted into a subcutaneous space of a patient. The reusable portion includes a reusable housing; and an actuator movable relative to the reusable housing. The reusable portion is operable to, in response to movement of the actuator relative to the reusable housing, automatically move the disposable portion from a stowed configuration in which the infusion cannula and the insertion needle are positioned within the reusable housing to an insertion configuration in which the infusion cannula and the insertion needle extend from the reusable housing. The reusable portion is further operable to thereafter automatically move the insertion needle from the insertion configuration to a retracted configuration in which the insertion needle is positioned within the disposable housing.

According to another exemplary embodiment of the present disclosure, an inserter device is disclosed including a reusable portion configured to detachably couple to a disposable portion. The disposable portion includes a disposable housing, an insertion needle, and an infusion cannula detachably coupled to the disposable housing and the insertion needle, and the infusion cannula configured to be inserted into a subcutaneous space of a patient. The reusable portion includes a reusable housingand an actuator movable relative to the reusable housing. The reusable portion is operable to, in response to movement of the actuator relative to the reusable housing, automatically move the disposable portion from a stowed configuration in which the infusion cannula and the insertion needle are positioned within the reusable housing to an insertion configuration in which the infusion cannula and the insertion needle extend from the reusable housing. The reusable portion is further operable to thereafter automatically move the insertion needle from the insertion configuration to a retracted configuration in which the insertion needle is positioned within the disposable housing.

According to yet another exemplary embodiment of the present disclosure, an inserter device is disclosed including a housing; an insertion needle; and a shift head infusion assembly detachably coupled to the housing and the insertion needle. The shift head infusion assembly includes an infusion head and an infusion cannula coupled to and movable with the infusion head. The infusion cannula is configured to be inserted into a subcutaneous space of a patient. The inserter device further includes an actuator that is movable relative to the housing. The inserter device is operable to, in response to movement of the actuator relative to the housing, automatically move the shift head infusion assembly from a stowed configuration in which the infusion cannula and the insertion needle are positioned within the housing to an insertion configuration in which the infusion cannula and the insertion needle extend from the housing. The inserter device is further operable to thereafter automatically move the insertion needle from the insertion configuration to a retracted configuration in which the insertion needle is positioned within the housing.

### BRIEF DESCRIPTION OF THE DRAWINGS

The above-mentioned and other features and advantages of this disclosure, and the manner of attaining them, will become more apparent and will be better understood by reference to the following description of embodiments of the invention taken in conjunction with the accompanying drawings, wherein:

FIG. 1 is a schematic view of a prior art example of an inserter device that shifts an infusion set head.

FIG. 2 is a schematic view of a prior art example of an inserter device that shifts a cannula.

FIG. 3 is a top perspective view of an inserter device according to an embodiment of the present disclosure.

FIG. 4 is an exploded bottom perspective view of the inserter device of FIG. 3.

FIG. 5 is an exploded top perspective view of a reusable portion of the inserter device of FIG. 3.

FIG. 6 is an exploded top perspective view of a disposable portion of the inserter device of FIG. 3.

FIG. 7 is a perspective sectional view of the disposable portion of FIG. 6 being advanced toward the reusable portion of FIG. 5.

FIG. 8 is a perspective sectional view of the disposable portion of FIG. 6 being further advanced toward the reusable portion of FIG. 5.

FIG. 9 is an enlarged perspective sectional view of the area 9-9 of FIG. 8, illustrating a first attachment mechanism before attaching the disposable portion to the reusable portion.

FIG. 10 is another enlarged perspective sectional view like FIG. 9, although illustrating the first attachment mechanism upon attaching the disposable portion to the reusable portion.

FIG. 11 is a perspective sectional view of the disposable portion of FIG. 6 being further advanced toward the reusable portion of FIG. 5.

FIG. 12 is an enlarged perspective sectional view of the area 12-12 of FIG. 11, illustrating a second attachment mechanism upon attaching a needle assembly of the disposable portion to a retraction hub of the reusable portion.

FIG. 13 is another perspective sectional view of the disposable portion of FIG. 6 being further advanced toward the reusable portion of FIG. 5.

FIG. 14 is an enlarged perspective sectional view of the area 14-14 of FIG. 13, illustrating a third attachment mechanism before attaching a push plate of the reusable portion to the retraction hub.

FIG. 15 is another enlarged perspective sectional view like FIG. 14, although illustrating the third attachment mechanism upon attaching the push plate to the retraction hub.

FIG. 16 is an enlarged perspective sectional view of the inserter device of FIG. 3 illustrating an actuator and a shift plate being moved to a set configuration.

FIG. 17 is an enlarged side sectional view of the inserter device of FIG. 3 illustrating the actuator and the shift plate being moved to the set configuration.

FIG. 18 is another enlarged side sectional view of the inserter device of FIG. 3 illustrating the actuator and the shift plate being moved to the set configuration.

FIG. 19 is another enlarged perspective sectional view of the inserter device of FIG. 3 illustrating the actuator and the shift plate being moved to the set configuration.

FIG. 20 is a perspective sectional view of the inserter device of FIG. 3 illustrating the actuator and the shift plate in the set configuration.

FIG. 21 is an enlarged perspective sectional view of the area 21-21 of FIG. 20, illustrating the shift plate securing the third attachment mechanism.

FIG. 22 is a side perspective view of the inserter device of FIG. 3 in a loaded or ready state.

FIG. 23 is an enlarged perspective sectional view of the area 23-23 of FIG. 22, illustrating the second attachment mechanism securing the needle assembly of the disposable portion to the retraction hub of the reusable portion.

FIG. 24 is an enlarged perspective sectional view of the inserter device of FIG. 3, illustrating a pedestal being detached from the device.

FIG. 25 is a bottom perspective view of the inserter device of FIG. 3, illustrating the pedestal being detached from the device.

FIG. 26 is a perspective sectional view of the inserter device of FIG. 3 moving from a stowed configuration toward an insertion configuration.

FIG. 27 is a perspective sectional view of the inserter device of FIG. 3 moving further toward the insertion configuration.

FIG. 28 is an enlarged perspective sectional view of the inserter device of FIG. 3, illustrating the shift plate releasing the third attachment mechanism.

FIG. 29 is a perspective sectional view of the inserter device of FIG. 3 moving from the insertion configuration toward a retracted configuration.

FIG. 30 is a bottom perspective view of the inserter device of FIG. 3, illustrating the disposable portion of FIG. 6 being rotated relative to the reusable portion of FIG. 5.

FIG. 31 is an enlarged perspective view of the inserter device of FIG. 3, illustrating the first attachment mechanism of FIGS. 9 and 10 being disengaged.

FIG. 32 is an enlarged perspective view of the inserter device of FIG. 3, illustrating the second attachment mechanism of FIG. 12 being disengaged.

FIG. 33 is a perspective sectional view of the disposable portion of FIG. 6 being detached from the reusable portion of FIG. 5.

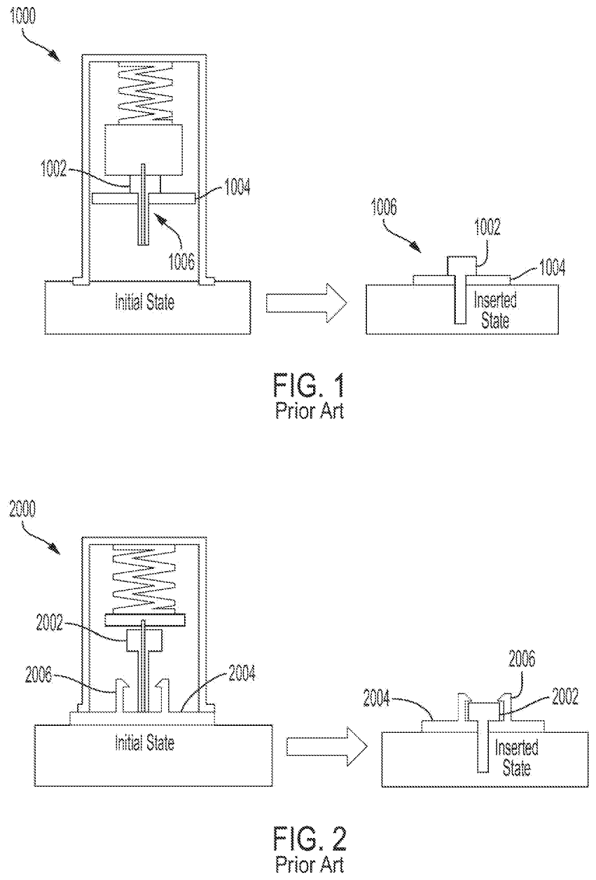

Corresponding reference characters indicate corresponding parts throughout the several views. The exemplifications set out herein illustrate exemplary embodiments of the invention and such exemplifications are not to be construed as limiting the scope of the invention in any manner.

### DETAILED DESCRIPTION

For the purpose of promoting an understanding of the principles of the invention, reference will now be made to the embodiments illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. One embodiment of the invention is shown in great detail, although it will be apparent to those skilled in the relevant art that some features that are not relevant to the present invention may not be shown for the sake of clarity.

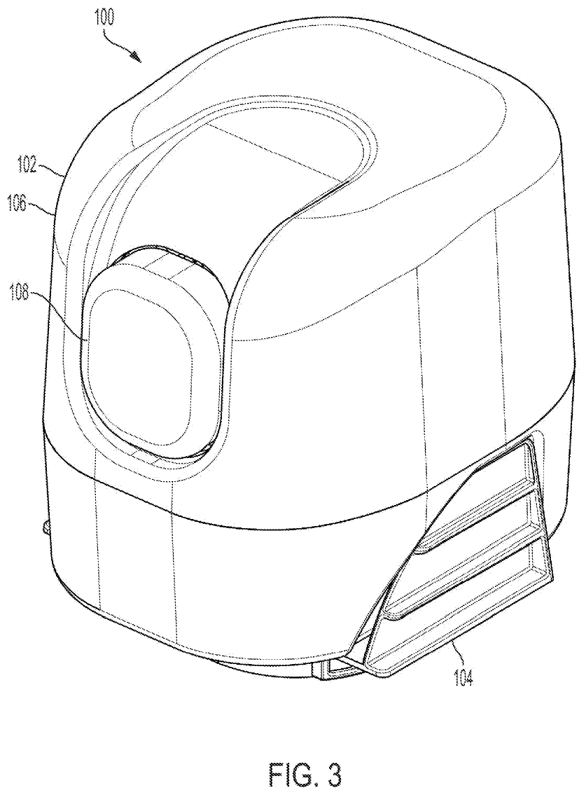

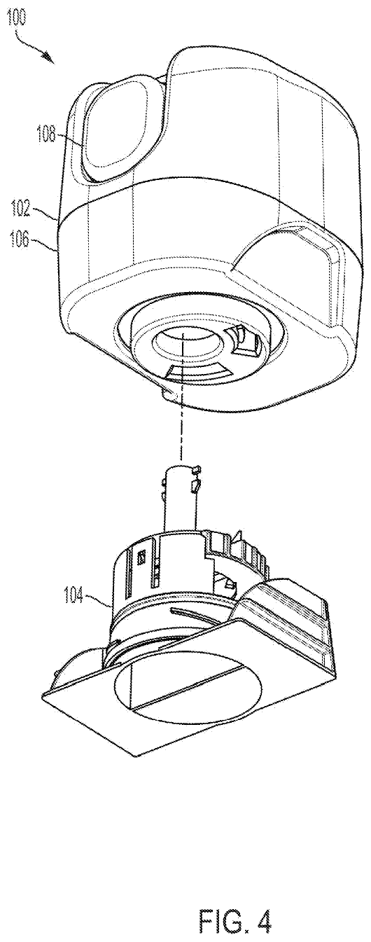

FIGS. 3 and 4 illustrate a shift head inserter device 100 according to an exemplary embodiment of the present disclosure. The inserter device 100 provides automatic insertion of an insertion needle and infusion cannula (both shown elsewhere) and subsequent automatic retraction of the insertion needle. In addition, the inserter device 100 is partially reusable.

With continued reference to FIGS. 3 and 4, the inserter device 100 generally includes a reusable portion 102, which may also be referred to as a durable portion, that detachably receives a disposable portion 104, which may also be referred to as a single-use portion. The reusable portion 102 includes a reusable housing 106 that movably supports an actuator 108, which may also be referred to as a button. The actuator 108 is actuatable to cause the reusable portion 102 to actuate the disposable portion 104 and thereby attach an infusion head assembly (shown elsewhere) to the skin of a patient. As described in further detail below, an infusion cannula of the infusion head assembly enters the subcutaneous space of the patient and is thereby configured to deliver a drug to the patient.

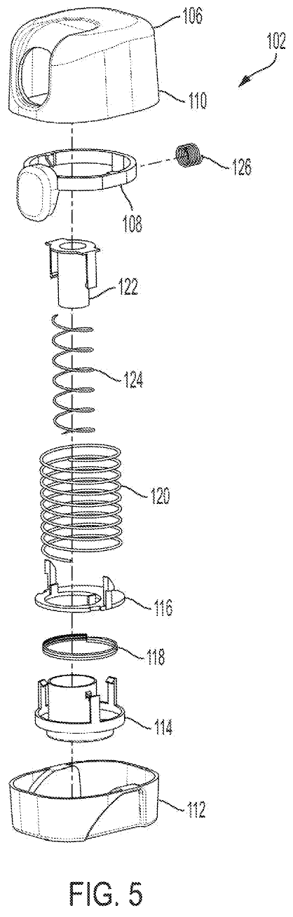

FIG. 5 illustrates the reusable portion 102 in further detail. The reusable housing 106 of the reusable portion 102 includes an upper housing 110 and a lower housing 112 that carry various internal components. More specifically, the lower housing 112 movably supports a push plate 114, and the push plate 114 in turn movably supports a shift plate 116. A shift mechanism 118, for example a compression spring, is disposed between and urges apart the push plate 114 and the shift plate 116. An insertion mechanism 120, for example a compression spring, is disposed between the shift plate 116 and the upper housing 110 and urges the shift plate 116 distally relative to the upper housing 110. The push plate 114 also movably supports a retraction hub 122. A retraction mechanism 124, for example a compression spring, is disposed between and urges apart the push plate 114 and the retraction hub 122. The upper housing 110 movably supports the actuator 108, and a setting mechanism 126, for example a compression spring, is disposed between and urges the actuator 108 in a transverse direction relative to the upper housing 110. Operation of these components of the reusable portion 102 is described in further detail below.

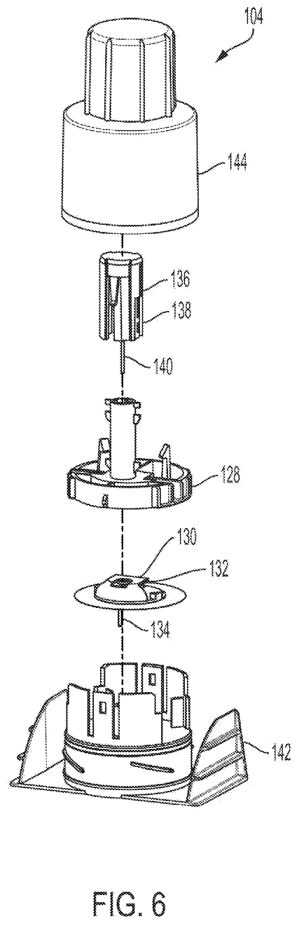

FIG. 6 illustrates the disposable portion 104 in further detail. The disposable portion 104 includes a disposable housing 128 that detachably carries an infusion head assembly 130. The infusion head assembly 130 includes an infusion head 132 and an infusion cannula 134. The disposable housing 128 movably supports a needle assembly 136. The needle assembly 136 includes a needle carrier 138 that supports an insertion needle 140, which initially extends through the infusion head 132 and the infusion cannula 134. The disposable housing 128 also detachably carries a distal pedestal 142, which in turn detachably carries a proximal cover 144. The proximal cover 144 initially preserves sterility of the insertion needle 140. Operation of these components of the disposable portion 104 is described in further detail below.

A method of coupling the disposable portion 104 to the reusable portion 102 is described in further detail below. Before coupling the disposable portion 104 to the reusable portion 102 according to this method, however, the cover 144 of the disposable portion 104 is detached from the pedestal 142, for example, by unscrewing the cover 144 from the pedestal 142.

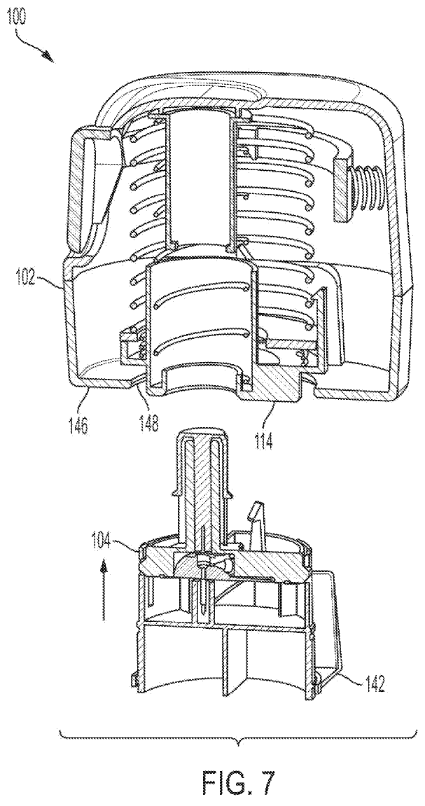

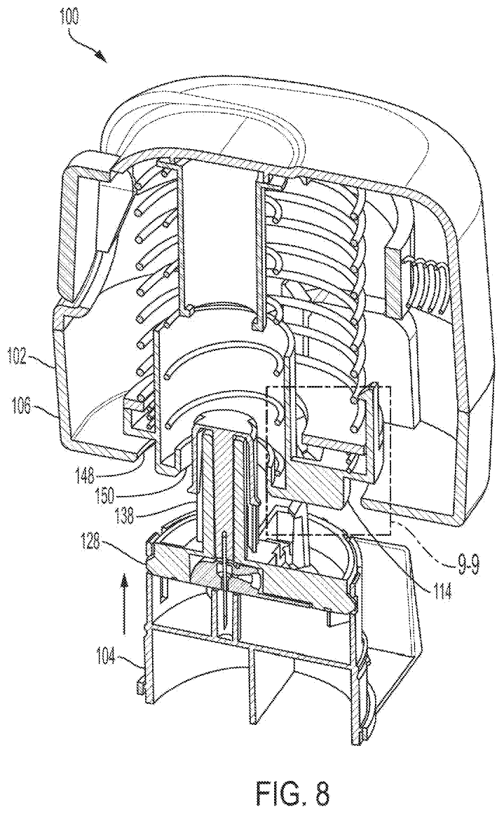

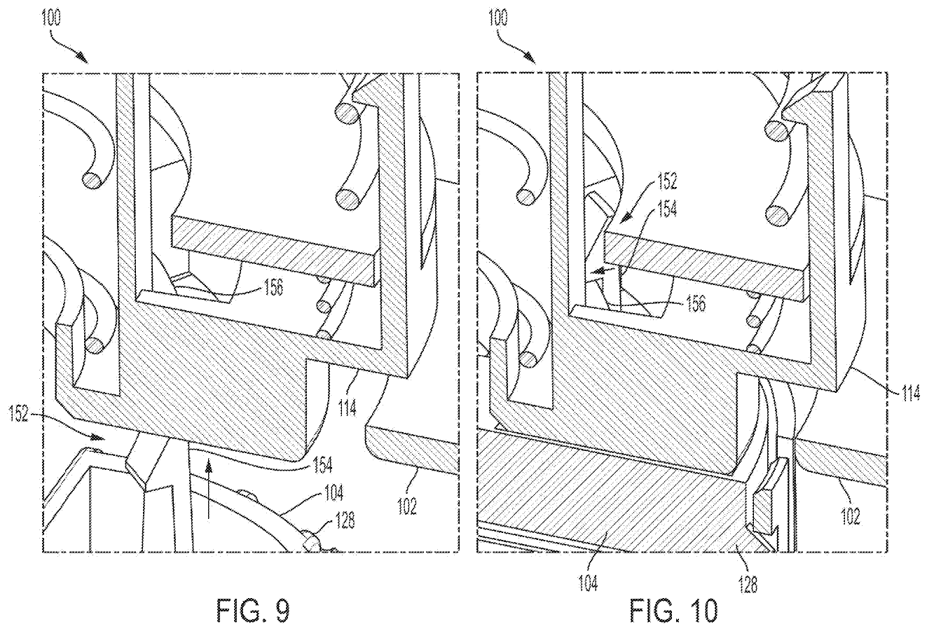

FIGS. 7-23 illustrate the method of coupling the disposable portion 104 to the reusable portion 102. First, after the cover 144 (shown elsewhere) is detached from the pedestal 142, the disposable portion 104 is positioned adjacent the reusable portion 102. More specifically and referring to FIG. 7, the disposable portion 104 is positioned adjacent to a distal end 146, a distal opening 148, and the push plate 114 of the reusable portion 102. With continued reference to FIG. 7, the disposable portion 104 is then advanced axially toward the reusable portion 102. Next and referring to FIG. 8, the disposable portion 104 is further advanced toward the reusable portion 102 such that the disposable portion 104 partially enters the reusable portion 102. More specifically, the needle carrier 138 and the disposable housing 128 extend into the distal opening 148 of the reusable housing 106 and a distal opening 150 of the push plate 114. Referring to FIGS. 9 and 10, the disposable portion 104 is then further advanced toward the reusable portion 102 (FIG. 9) to attach the disposable portion 104 to the reusable portion 102 (FIG. 10). More specifically, a first attachment mechanism 152 attaches the disposable portion 104 to the reusable portion 102. Illustratively, the first attachment mechanism 152 include a flexible snap or arm 154 of the disposable housing 128 that latches to a ledge 156 of the push plate 114. In other embodiments, the first attachment mechanism 152 may take other forms.

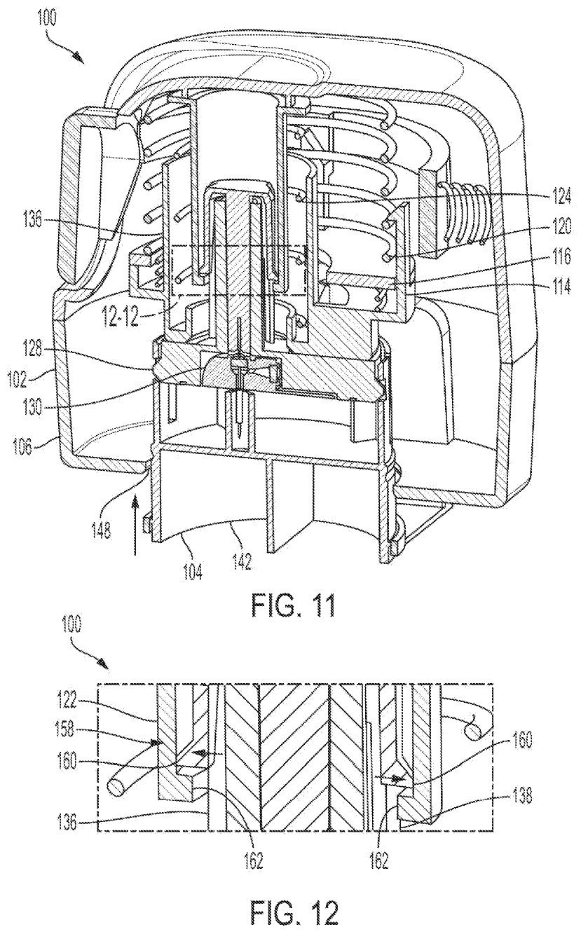

Next and referring to FIG. 11, the disposable portion 104 is further advanced toward the reusable portion 102 such that the disposable portion 104 further enters the reusable portion 102. More specifically, the needle assembly 136, the disposable housing 128, and the infusion head assembly 130 are disposed within the reusable housing 106 and the pedestal 142 extends through the distal opening 148 of the reusable housing 106. This advancement also causes the push plate 114 and the shift plate 116 to move proximally within the reusable housing 106, which in turn loads the insertion mechanism 120 and the retraction mechanism 124. Referring to FIG. 12, this advancement further causes the needle assembly 136 to attach to the retraction hub 122. More specifically, a second attachment mechanism 158 attaches the needle assembly 136 to the retraction hub 122. Illustratively, the second attachment mechanism 158 may include one or more flexible snaps or arms 160 (for example, two arms 160) of the needle carrier 138 that each latch above a ledge 162 of the retraction hub 122. In other embodiments, the second attachment mechanism 158 may take other forms.

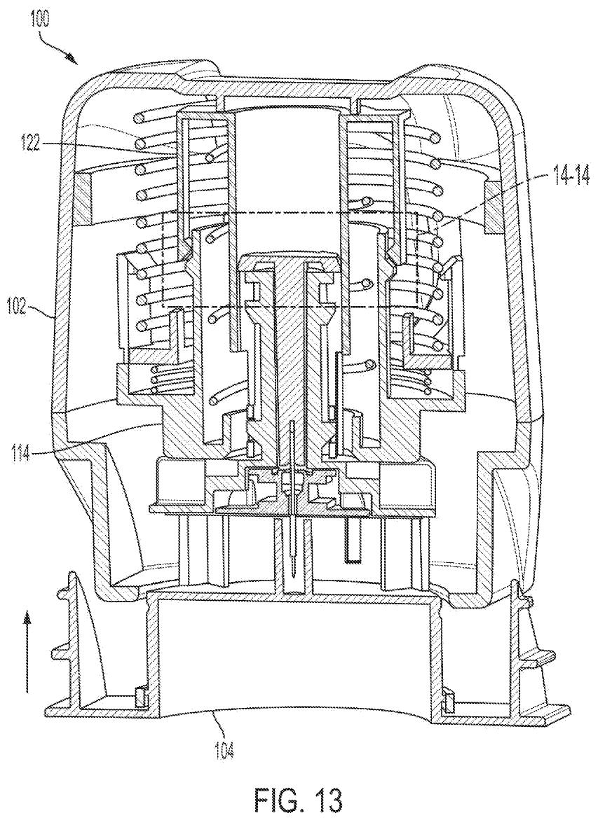

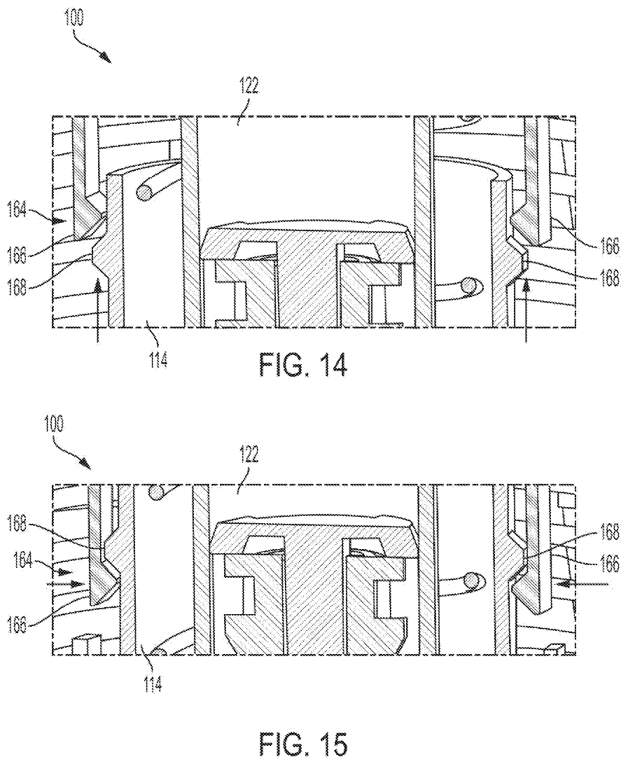

Referring to FIGS. 13-15, further advancement of the disposable portion 104 toward the reusable portion 102 as described above (and shown in FIGS. 13 and 14) also causes the push plate 114 to attach to the retraction hub 122 (FIG. 15). More specifically, a third attachment mechanism 164 attaches the push plate 114 to the retraction hub 122. Illustratively, the third attachment mechanism 164 may include one or more flexible snaps or arms 166 (for example, two arms 166) of the retraction hub 122 that each latch below a ledge 168 of the push plate 114. In other embodiments, the third attachment mechanism 164 may take other forms.

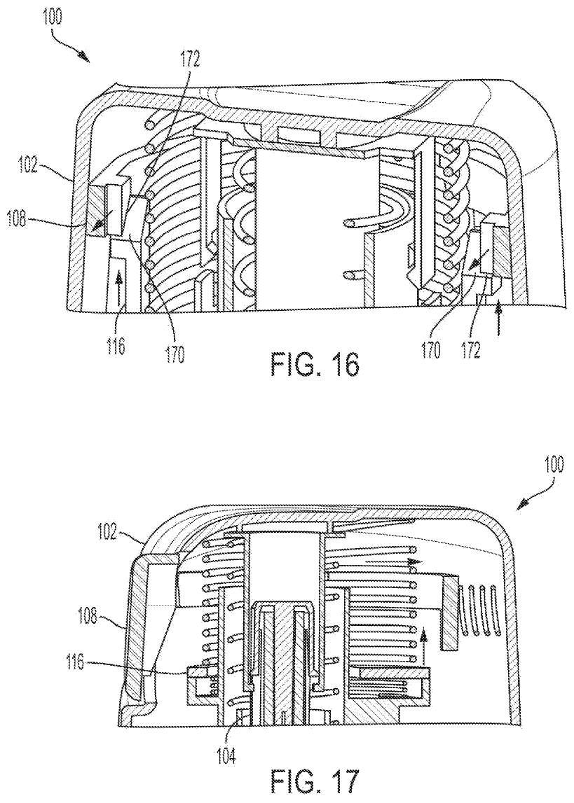

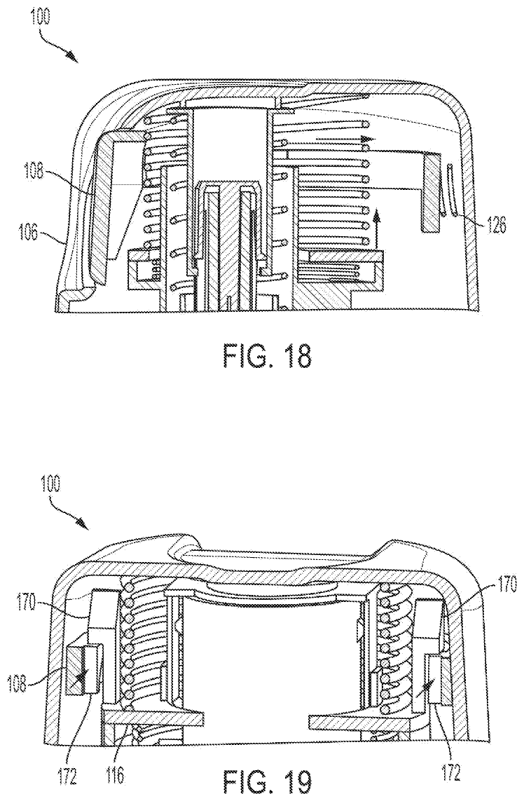

Referring to FIGS. 16 and 17, the disposable portion 104 (FIG. 17) is further advanced toward the reusable portion 102 to set the actuator 108 and the shift plate 116. More specifically, the disposable portion 104 is further advanced toward the reusable portion 102 to cause one or more ramps 170 (FIG. 16—for example, two ramps 170) of the shift plate 116 to each slidably engage a ramp 172 (FIG. 16) of the actuator 108. This interaction between the ramps 170, 172 causes, as shown in FIG. 18, the actuator 108 to move in a transverse direction relative the reusable housing 106, which in turn loads the setting mechanism 126. The ramps 172 of the actuator 108 subsequently clear the ramps 170 of the shift plate 116, and the setting mechanism 126 releases stored energy to move the actuator 108 in the opposite transverse direction. As a result and as shown in FIG. 19, the ramps 172 of the actuator 108 are positioned under the ramps 170 of the shift plate 116, and the actuator 108 thereby holds the shift plate 116 in an elevated or set configuration.

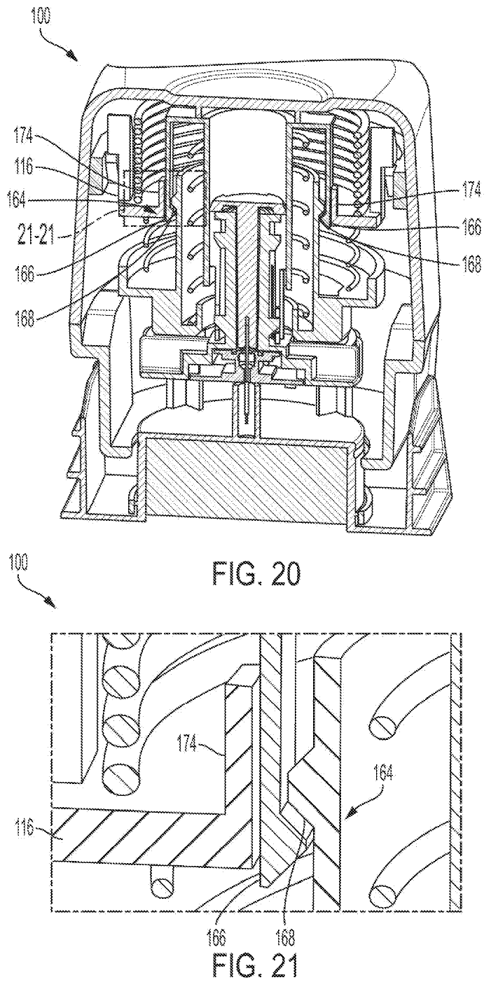

Referring to FIGS. 20 and 21, in the set configuration the shift plate 116 secures the third attachment mechanism 164. More specifically, one or more walls 174 (for example, two walls 174) of the shift plate 116 each contact one of the flexible arms 166 of the retraction hub 122 to hold the arms 166 below the ledges 168 of the push plate 114.

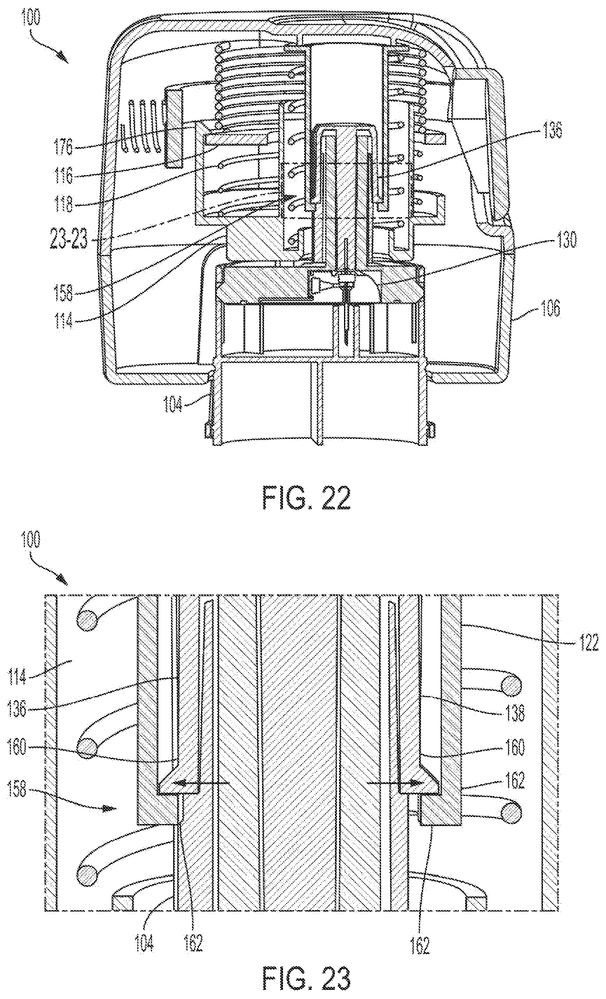

Referring to FIG. 22, in the set configuration the shift mechanism 118 releases stored energy to move the push plate 114 distally relative to the shift plate 116. One or more stops 176 (for example, two stops 176, one stop 176 being visible in FIG. 22) of the push plate 114 provide a limit to this relative motion between the push plate 114 and the shift plate 116. In addition, in the set configuration the shift mechanism 118 releases stored energy to secure the second attachment mechanism 158. More specifically, the shift mechanism 118 releases energy to move the push plate 114 and the disposable portion 104, including the needle assembly 136, distally relative to the shift plate 116, and, as shown in FIG. 23, the arms 160 of the needle carrier 138 are thereby seated atop the ledges 162 of the retraction hub 122.

As a result of the above actions, the inserter device 100 occupies a ready state. In the ready state and as shown in FIG. 22, the infusion head assembly 130 and the needle assembly 136 occupy a stowed configuration and are positioned in the reusable housing 106. The inserter device 100 may be maintained or carried in the ready state until the infusion head assembly 130 is to be attached to a patient.

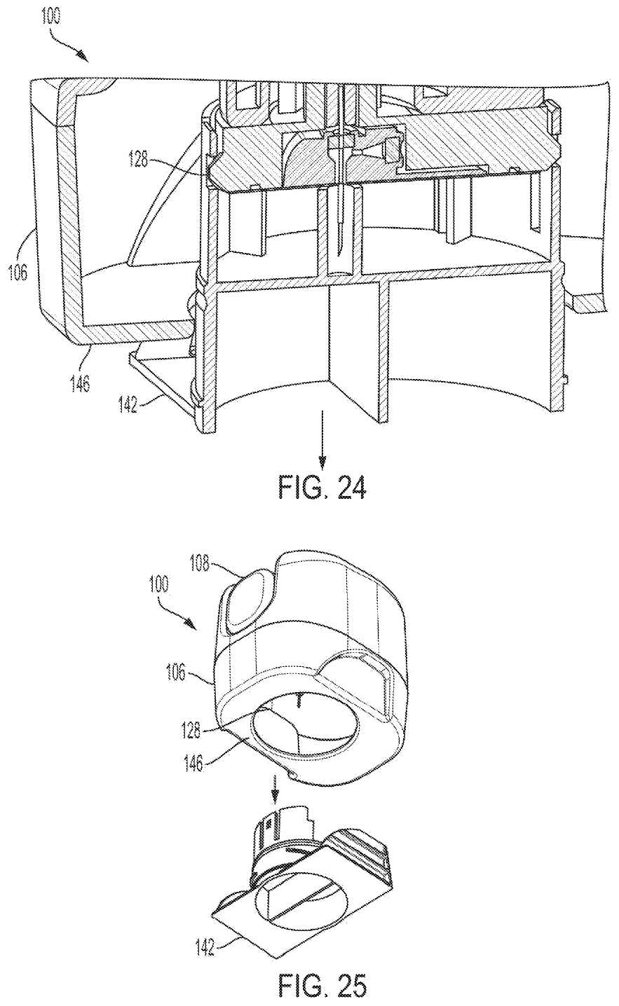

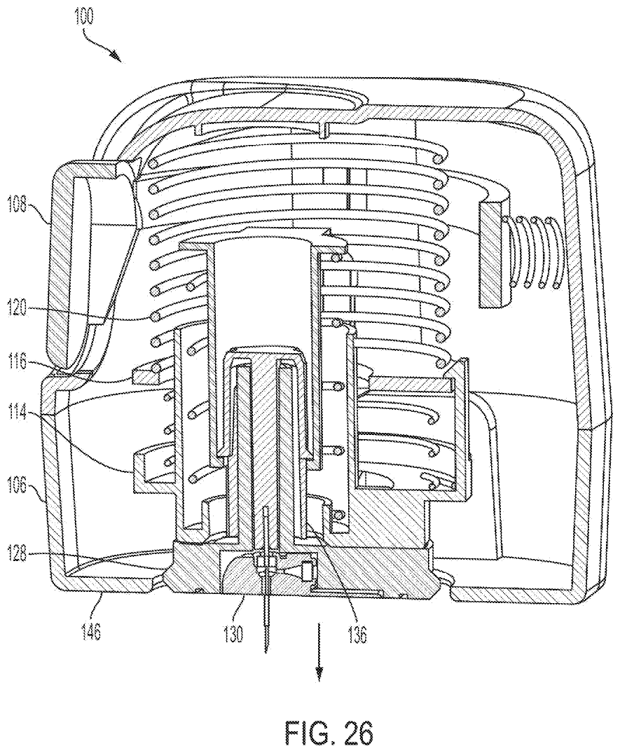

FIGS. 24-29 illustrate a method of using the inserter device 100 to attach the infusion head assembly 130 to the skin of a patient. First and as shown in FIGS. 24 and 25, when the inserter device 100 is in the ready state, the pedestal 142 is detached from the remainder of the device 100. More specifically, the pedestal 142 is axially pulled apart from the disposable housing 128. Next, the distal end 146 of the reusable housing 106 is held against the skin of the patient. A user (for example, the patient, a medical practitioner, or another person) applies a force to the actuator 108 such that the actuator 108 is urged in the transverse direction. As shown in FIG. 26, this action causes the actuator 108 to release the shift plate 116, which permits the insertion mechanism 120 to release stored energy. As a result, the insertion mechanism 120 automatically moves the shift plate 116, the push plate 114, and the disposable housing 128 distally relative to the reusable housing 106, and the infusion head assembly 130 and the needle assembly 136 move from the stowed configuration toward an insertion configuration.

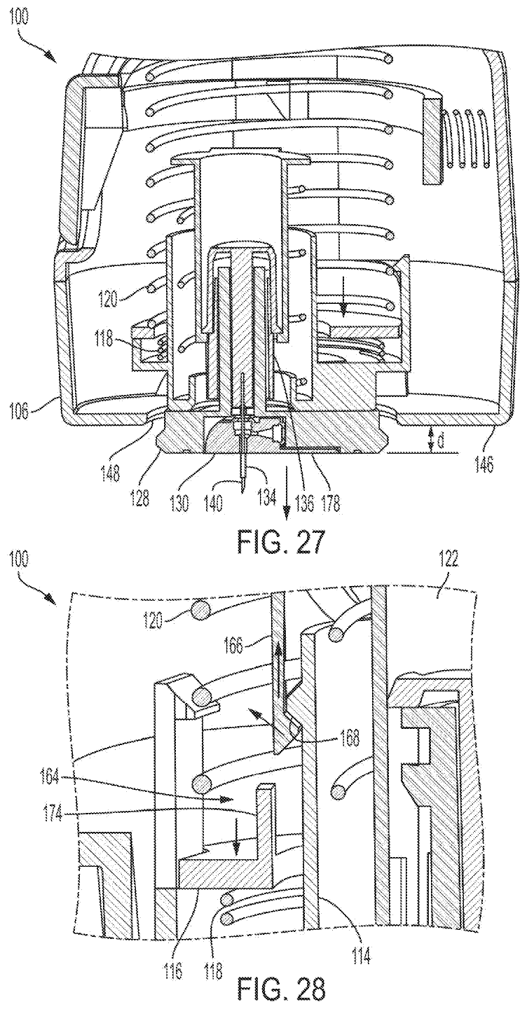

As shown in FIG. 27, the insertion mechanism 120 continues to release stored energy until the infusion head assembly 130 and the needle assembly 136 reach the insertion configuration. In the insertion configuration, the disposable housing 128, the infusion head assembly 130, and the needle assembly 136 extend outwardly from the distal opening 148 of the reusable housing 106. As a result, the insertion needle 140 pierces the skin of the patient and the infusion cannula 134 enters the subcutaneous space of the patient. Additionally, in the insertion configuration the disposable housing 128 may extend a distance d below the distal end 146 of the reusable housing 106 (for example, 1-2 mm). This facilitates compressing the skin of the patient and securing the infusion head assembly 130 to the skin via an adhesive patch or surface 178 carried on the distal surface of the infusion head assembly 130. In some embodiments, the adhesive surface 178 is the only adhesive surface of the device 100. More specifically and advantageously, the device 100 lacks a second adhesive surface or layer that is initially secured to the skin of the patient and subsequently secured to the infusion head assembly 130 via the adhesive surface 178.

Referring to FIGS. 27 and 28, as the insertion mechanism 120 continues to release stored energy as described above, the insertion mechanism 120 moves the shift plate 116 distally and toward the push plate 114. This action loads the shift mechanism 118. As shown in FIG. 28, this also causes the shift plate 116 to release the third attachment mechanism 164. More specifically, the walls 174 of the shift plate 116 move apart from the flexible arms 166 of the retraction hub 122, which permits the flexible arms 166 to move apart from the ledges 168 of the push plate 114.

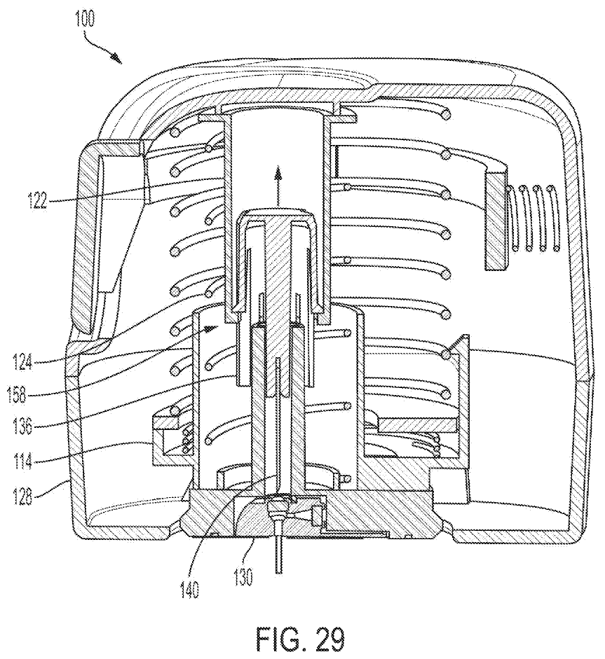

Disengagement of the third attachment mechanism 164 permits, as shown in FIG. 29, the retraction mechanism 124 to release stored energy and automatically move the retraction hub 122 proximally relative to the push plate 114. The retraction hub 122, via the second attachment mechanism 158, also moves the needle assembly 136 proximally relative to the disposable housing 128. As a result, the device 100 occupies a retracted configuration in which the insertion needle 140 is positioned and shielded within the disposable housing 128. The inserter device 100 is then moved away from the skin of the patient to leave the infusion head assembly 130 secured to the skin of the patient.

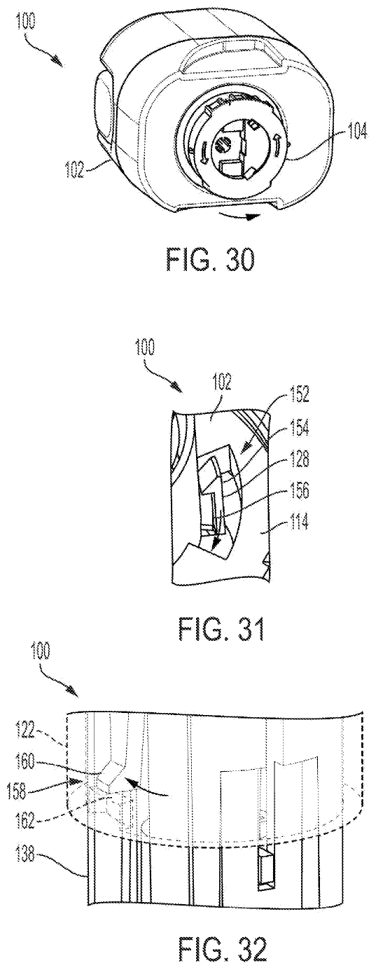

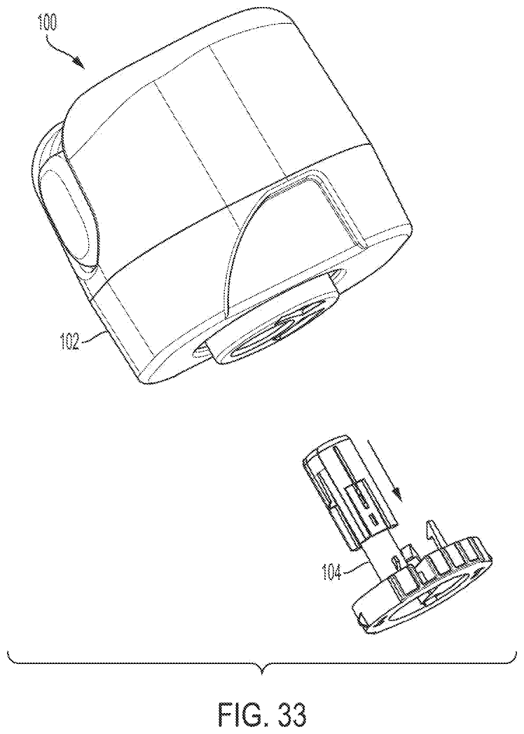

FIGS. 30-33 illustrate a method of detaching the disposable portion 104 from the reusable portion 102. First and as shown in FIG. 30, the disposable housing 128 is rotated relative to the reusable housing 106. As shown in FIG. 31, this action causes the first attachment mechanism 152 to disengage. More specifically, the arm 154 of the disposable housing 128 moves apart from the ledge 156 of the push plate 114. As shown in FIG. 32, this action also causes the second attachment mechanism 158 to disengage. More specifically, the arms 160 of the needle carrier 138 move apart from the ledges 162 of the retraction hub 122. As shown in FIG. 33, the disposable portion 104 is then pulled axially away from the reusable portion 102 while the insertion needle 140 (shown elsewhere) remains in the retracted configuration. A fresh disposable portion 104 may then be coupled to the reusable portion 102 in the manner described above.

Inserter devices according to the present disclosure may be modified in various other manners. For example, the insertion mechanism 120, the retraction mechanism 124, the shift mechanism 118, and the setting mechanism 126 are illustrated as compressions springs. However, and as an exemplary modification, one or more of these mechanisms could be other compliant energy storing components (for example, flexible beams or the like). As another exemplary modification, inserter devices according to the present disclosure may be provided with one or more locks for selectively inhibiting actuation of the actuator. Such a lock may be any suitable device known in the art.

In the figures described above, the inserter device 100 is illustrated with an infusion head assembly 130 having a cannula 134 that is offset relative to the center of the inserter device 100. However, and as another exemplary modification, inserter devices according to the present disclosure can include other types of infusion head assemblies, including infusion head assemblies having cannulas that are aligned with the center of the inserter device.

Inserter devices according to the present disclosure may provide various additional advantages over other inserter devices. For example, a single action, specifically advancing the disposable portion 104 toward the reusable portion 102, both couples the disposable portion 104 to the reusable portion 102, resets the insertion mechanism 120, the retraction mechanism 124, and the shift mechanism 118, and sets the actuator 108. As another example, the disposable portion 104 may be used to attach the infusion head assembly 130 to the patient without the reusable portion 102. More specifically and referring to FIG. 6, the cover 144 may be detached from the pedestal 142, the pedestal 142 may be detached from the reusable housing 106, and a compressive force may be applied to the needle assembly 136 to pierce the skin of the patient with the insertion needle 140 and attach the infusion head assembly 130 to the patient. The needle assembly 136 may then be pulled proximally away from the disposable housing 128 to move the insertion needle 140 to the retracted configuration. In this way, the patient is not exposed to the used insertion needle 140, which is shielded by the disposable portion 104 and can be discarded with the disposable portion 104.

While this invention has been described as having exemplary designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

1. An inserter device comprising:
a disposable portion comprising:
a disposable housing;
an insertion needle;
an infusion cannula detachably coupled to the disposable housing and the insertion needle, the infusion cannula configured to be inserted into a subcutaneous space of a patient;
a reusable portion configured to detachably couple to the disposable portion, the reusable portion comprising:
a reusable housing; and
an actuator movable relative to the reusable housing;
wherein the reusable portion is operable to, in response to movement of the actuator relative to the reusable housing, automatically move the disposable portion from a stowed configuration in which the infusion cannula and the insertion needle are positioned within the reusable housing to an insertion configuration in which the infusion cannula and the insertion needle extend from the reusable housing;
wherein the reusable portion is further operable to thereafter automatically move the insertion needle from the insertion configuration to a retracted configuration in which the insertion needle is positioned within the disposable housing.

2. The inserter device of claim 1, wherein the reusable portion further comprises a push plate movable relative to the reusable housing and configured to detachably couple to the disposable portion, wherein the reusable portion is operable to, in response to movement of the actuator relative to the reusable housing, automatically move the push plate and the disposable portion from the stowed configuration to the insertion configuration.

3. The inserter device of claim 2, wherein the reusable portion further comprises:
an insertion mechanism operable to, in response to movement of the actuator relative to the reusable housing, automatically move the push plate and the disposable portion from the stowed configuration to the insertion configuration;
a retraction mechanism operable to automatically move the insertion needle from the insertion configuration to the retracted configuration; and
a shift mechanism operable to inhibit operation of the retraction mechanism in the stowed configuration and permit operation of the retraction mechanism in the insertion configuration.

4. The inserter device of claim 3, wherein the reusable portion further comprises a shift plate movable relative to the push plate, wherein the shift mechanism comprises a spring that urges the shift plate away from the push plate.

5. The inserter device of claim 4, wherein the reusable portion further comprises a retraction hub movable relative to the push plate, and the shift plate secures the retraction hub to the push plate to inhibit operation of the retraction mechanism in the stowed configuration.

6. The inserter device of claim 5, wherein the disposable portion comprises a needle assembly, the needle assembly comprising:
the insertion needle; and
a needle carrier movable relative to the disposable housing and configured to detachably couple to the retraction hub, the insertion needle being fixed relative to the needle carrier.

7. The inserter device of claim 6, wherein the retraction mechanism is operable to automatically move the retraction hub, the needle carrier, and the insertion needle from the insertion configuration to the retracted configuration.

8. The inserter device of claim 5, wherein the shift plate comprises a wall configured to abuttingly engage the retraction hub and thereby secure the retraction hub to the push plate to inhibit operation of the retraction mechanism in the stowed configuration.

9. The inserter device of claim 4, wherein the reusable portion further comprises a stop to limit motion of the shift plate relative to the push plate.

10. The inserter device of claim 4, wherein the shift plate comprises a ramp configured to slide against the actuator.

11. The inserter device of claim 3, wherein the shift mechanism comprises a compression spring.

12. The inserter device of claim 3, wherein the disposable portion further comprises an infusion head coupled to the infusion cannula, the infusion head comprising an adhesive surface configured to couple the infusion head to the patient when the infusion cannula is inserted into the subcutaneous space of the patient.

13. The inserter device of claim 3, wherein the disposable portion further comprises a cover detachably coupled to the disposable housing and preserving sterility of the insertion needle, the cover configured to be detached from the disposable housing before coupling the disposable portion to the reusable portion.

14. An inserter device comprising:
a reusable portion configured to detachably couple to a disposable portion, the disposable portion comprising a disposable housing, an insertion needle, and an infusion cannula detachably coupled to the disposable housing and the insertion needle, the infusion cannula configured to be inserted into a subcutaneous space of a patient, the reusable portion comprising:
a reusable housing; and
an actuator movable relative to the reusable housing;
wherein the reusable portion is operable to, in response to movement of the actuator relative to the reusable housing, automatically move the disposable portion from a stowed configuration in which the infusion cannula and the insertion needle are positioned within the reusable housing to an insertion configuration in which the infusion cannula and the insertion needle extend from the reusable housing;
wherein the reusable portion is further operable to thereafter automatically move the insertion needle from the insertion configuration to a retracted configuration in which the insertion needle is positioned within the disposable housing.

15. The inserter device of claim 14, wherein the reusable portion further comprises:
a push plate movable relative to the reusable housing and configured to detachably couple to the disposable portion;
a shift plate movable relative to the push plate;
a retraction hub movable relative to the push plate and configured to detachably couple to the disposable portion;
an insertion mechanism operable to, in response to movement of the actuator relative to the reusable housing, automatically move the push plate, the shift plate, the retraction hub, and the disposable portion from the stowed configuration to the insertion configuration;
a retraction mechanism operable to automatically move the retraction hub and the insertion needle relative to the push plate and from the insertion configuration to the retracted configuration; and
a shift mechanism operable to secure the retraction hub to the push plate via the shift plate in the stowed configuration and thereby inhibit operation of the retraction mechanism in the stowed configuration.

16. The inserter device of claim 15, wherein the shift mechanism comprises a compression spring that holds the shift plate in abutting engagement with the retraction hub to secure the retraction hub to the push plate in the stowed configuration, the compression spring thereby inhibiting operation of the retraction mechanism in the stowed configuration.

17. The inserter device of claim 15, wherein the reusable portion further comprises a stop to limit motion of the shift plate relative to the push plate.

18. The inserter device of claim 15, wherein the shift plate comprises a ramp configured to slide against the actuator.

19. An inserter device comprising:
a housing;
an insertion needle;
a shift head infusion assembly detachably coupled to the housing and the insertion needle, the shift head infusion assembly comprising an infusion head and an infusion cannula coupled to and movable with the infusion head, the infusion cannula configured to be inserted into a subcutaneous space of a patient; and
an actuator movable relative to the housing;
wherein the inserter device is operable to, in response to movement of the actuator relative to the housing, automatically move the shift head infusion assembly from a stowed configuration in which the infusion cannula and the insertion needle are positioned within the housing to an insertion configuration in which the infusion cannula and the insertion needle extend from the housing;
wherein the inserter device is further operable to thereafter automatically move the insertion needle from the insertion configuration to a retracted configuration in which the insertion needle is positioned within the housing.
